(19) United States
(12) Patent Application Publication
(10) Pub. No.: US 2003/0065235 A1
(43) Pub. Date: Apr. 3, 2003

(54) Oxidative dehydrogenation of alkanes to olefins using an oxide surface

(76) Inventors: Joe D. Allison, Ponca City, OK (US); Lisa M. Budin, Ponca City, OK (US)

Correspondence Address: DAVID W. WESTPHAL, CONOCO PHILLIPS, P.O. BOX 1267, PONCA CITY, OK, 74602-1267, US

(21) Appl. No.: 10/118,421

(22) Filed: Apr. 8, 2002

Related U.S. Application Data
(60) Provisional application No. 60/324,346, filed on Sep. 24, 2001.

(51) Int. Cl. C07C 5/373

(52) U.S. Cl. 585/656; 585/658; 585/660

(57) ABSTRACT

A catalyst useful for the production of olefins from alkanes via oxidative dehydrogenation (ODH) is disclosed. The catalyst includes a base metal, metal oxide, or combination thereof and a refractory support. The base metal is selected from the group containing Group IB-VIIB metals, Group IIIA-VA metals, Lanthanide metals, iron, cobalt, and nickel. The metal oxide is selected from the group containing alumina, stabilized aluminas, zirconia, stabilized zirconias, titania, ytteria, silica, niobia, and vanadia. The catalyst does not contain any precious metals; it is activated by higher preheat temperatures. As a result, similar conversions are achieved at a considerably lower catalyst cost.

### TECHNICAL FIELD OF THE INVENTION

This invention generally relates to the conversion of alkanes to alkenes. More specifically, the invention relates to employing oxidative dehydrogenation (ODH) to convert alkanes to alkenes. Still more specifically, the invention relates to non-precious metal catalysts used in ODH.

### BACKGROUND OF THE INVENTION

In the commercial production of plastics, elastomers, man-made fibers, adhesives, and surface coatings, a tremendous variety of polymers are used. There are many ways to classify these compounds. For example, polymers can be categorized according to whether they are formed through chain-growth or step-growth reactions. Alternatively, polymers can be divided between those that are soluble in selective solvents and can be reversibly softened by heat, known as thermoplastics, and those that form three-dimensional networks that are not soluble and cannot be softened by heat without decomposition, known as thermosets. Additionally, polymers can be classified as either made from modified natural compounds or made from entirely synthetic compounds.

A logical way to classify the major commercially employed polymers is to divide them by the composition of their monomers, the chains of linked repeating units that make up the macromolecules. Classified according to composition, industrial polymers are either carbon-chain polymers (also called vinyls) or heterochain polymers (also called noncarbon-chain, or nonvinyls). In carbon-chain polymers, as the name implies, the monomers are composed of linkages between carbon atoms; in heterochain polymers a number of other elements are linked together in the monomers, including oxygen, nitrogen, sulfur, and silicon.

By far the most important industrial polymers are polymerized olefins, which comprise virtually all commodity plastics. Olefins, also called alkenes, are unsaturated hydrocarbons (compounds containing hydrogen [H] and carbon [C]) whose molecules contain one or more pairs of carbon atoms linked together by a double bond. The olefins are classified in either or both of the following ways: (1) as cyclic or acyclic (aliphatic) olefins, in which the double bond is located between carbon atoms forming part of a cyclic (closed-ring) or an open-chain grouping, respectively, and (2) as monoolefins, diolefins, triolefins, etc., in which the number of double bonds per molecule is, respectively, one two, three, or some other number. Hence, olefins are highly desired for the production of plastics.

Generally, olefin molecules are commonly represented by the chemical formula CH₂═CHR, where C is a carbon atom, H is a hydrogen atom, and R is an atom or pendant molecular group of varying composition. The composition and structure of R determines which of the huge array of possible properties will be demonstrated by the polymer.

More specifically, acyclic monoolefins have the general formula CₙH₂ₙ, where n is an integer. Acyclic monoolefins are rare in nature but are formed in large quantities during the cracking of petroleum oils to gasoline. The lower monoolefins, i.e., ethylene, propylene, and butylene, have become the basis for the extensive petrochemicals industry. Most uses of these compounds involve reactions of the double bonds with other chemical agents. Acyclic diolefins, also known as acyclic dialkenes, or acyclic dienes, with the general formula CₙH₂₋₂, contain two double bonds; they undergo reactions similar to the monoolefins. The best-known dienes are butadiene and isoprene, used in the manufacture of synthetic rubber.

Olefins containing two to four carbon atoms per molecule are gaseous at ordinary temperatures and pressure; those containing five or more carbon atoms are usually liquid at ordinary temperatures. Additionally, olefins are only slightly soluble in water.

Olefins have traditionally been produced from alkanes by fluid catalytic cracking (FCC) or steam cracking, depending on the size of the alkanes. Heavy olefins are herein defined as containing at least five carbon atoms and are produced by FCC. Light olefins are defined herein as containing one to four carbon atoms and are produced by steam cracking. Alkanes are similar to alkenes, except that they are saturated hydrocarbons whose molecules contain carbon atoms linked together by single bonds. The simplest alkanes are methane (CH₄, the most abundant hydrocarbon), ethane (CH₃CH₃), and propane (CH₃CH₂CH₃). These three compounds exist in only one structure each. Higher members of the series, beginning with butane (CH₃CH₂CH₂CH₃), may be constructed in two or more different ways, depending on whether the carbon chain is straight or branched. Such compounds are called isomers; these are compounds with the same molecular formula but different arrangements of their atoms. As a result, they often have different chemical properties.

In the conversion of alkanes to alkenes, FCC is a catalytic process, while steam cracking is a direct, non-catalytic dehydrogenation process. FCC and steam cracking are known to have drawbacks. For example, the processes are endothermic, meaning that heat is absorbed by the reactions and the temperature of the reaction mixtures decline as the reactions proceed. This is known to lower the product yield, resulting in lower value products. In addition, in FCC, coke forms on the surface of the catalyst during the cracking processes, covering active sites and deactivating the catalyst. During regeneration, the coke is burned off the catalyst to restore its activity and to provide heat needed to drive the cracking.

This cycle is very stressful for the catalyst; temperatures fluctuate between extremes as coke is repeatedly deposited and burned off. Furthermore, the catalyst particles move at high speed through steel reactors and pipes, where wall contacts and interparticle contacts are impossible to avoid.

While it may be easy to dismiss catalyst damage and loss in less expensive catalysts, the catalysts used in FCC units are quite expensive. The expense stems from the use of precious metals. For example, a typical supported metal catalyst may cost in the range of $20-$40 per pound, of which the cost of the precious metals may be between 50-80%. Thus, for a reactor that uses 2 million pounds of catalyst, the total cost of the metals in the reactor is considerable. Further, because FCC and steam cracking units are large and require steam input, the overall processes are expensive.

As a result, because olefins comprise the most important building blocks in modern petrochemical industry, the development of alternate routes other than FCC and steam reforming have been explored. One such route is oxidative dehydrogenation (ODH). In ODH, an organic compound is dehydrogenated in the presence of oxygen. Oxygen may be fed to the reaction zone as pure oxygen, air, oxygen-enriched air, oxygen mixed with a diluent, and so forth. Oxygen in the desired amount may be added in the feed to the dehydrogenation zone and oxygen may also be added in increments to the dehydrogenation zone. However, catalysts for oxidative dehydrogenation are still being investigated and the development of more effective catalysts for ODH is highly desirable.

### SUMMARY OF THE INVENTION

The present invention provides a non-precious metal catalyst for use in ODH. ODH was chosen for alkane dehydrogenation because it overcomes thermodynamic limitations of olefin yield faced in direct dehydrogenation and rapid coking of the catalysts resulting in short catalyst life.

Although oxidative dehydrogenation usually involves the use of a catalyst, and is therefore literally a catalytic dehydrogenation, oxidative dehydrogenation (ODH) is distinct from what is normally called “catalytic dehydrogenation” in that the former involves the use of an oxidant, and the latter does not. In the disclosure herein, “oxidative dehydrogenation”, though employing a catalyst, will be understood as distinct from so-called “catalytic dehydrogenation” processes in that the latter do not involve the interaction of oxygen with the hydrocarbon feed.

In accordance with a preferred embodiment of the present invention, a catalyst for use in ODH processes includes a non-precious base metal, metal oxide, or combination thereof and a refractory support. A non-precious base metal, referred to sometimes herein as a “base metal,” is defined herein as a non-Group VIII metal (using the CAS naming convention), with the exception of iron, cobalt and nickel. Suitable non-precious base metals include Group IB-VIIB metals (CAS convention), Group IIIA-VA metals (CAS convention), Lanthanide metals, iron, cobalt and nickel. Suitable metal oxides include alumina, stabilized aluminas, zirconia, stabilized zirconias (PSZ), titania, ytteria, silica, niobia, and vanadia.

In accordance with another preferred embodiment of the present invention, a method for the production of olefins includes contacting a preheated alkane and oxygen stream with a catalyst containing a non-precious base metal, metal oxide, or combination thereof and a refractory support, sufficient to initiate the oxidative dehydrogenation of the alkane (the preheat temperature being between 300-700° C.), maintaining a contact time of the alkane with the catalyst for less than 200 milliseconds, and maintaining oxidative dehydrogenation favorable conditions.

### DETAILED DESCRIPTION OF THE PREFERRED EMBODIMENTS

The present invention relates to non-precious metals, metal oxides, or combinations thereof placed on refractory supports for converting alkanes to alkenes via ODH. Typical ODH catalysts contain a precious metal, such as platinum, which promotes alkane conversion. The present invention, however, does not contain any precious metals and is activated by higher preheat temperatures. As a result, similar conversions are achieved at a considerably lower catalyst cost.

In a preferred embodiment of the present invention, light alkanes and O₂ are converted to the corresponding alkenes using novel non-precious metal or metal oxide catalysts. Preferably, a millisecond contact time reactor is used. Use of a millisecond contact time reactor for the commercial scale conversion of light alkanes to corresponding alkenes will reduce capital investment and increase alkene production significantly. It has been discovered that ethylene yield of 55% or higher in a single pass through the catalyst bed is achievable. This technology has the potential to achieve yields above that of the conventional technology at a much lower cost. The need for steam addition, as is currently required in the conventional cracking technology, is also eliminated by the present process. However, in some embodiments, the use of steam may be preferred. There is minimal coking in the present process and therefore little unit down time and loss of valuable hydrocarbon feedstock. Furthermore, the present novel catalysts improve the selectivity of the process to the desired alkene. In addition, the carbon oxide product that is produced at low levels is preferably primarily CO, rather than CO₂, and is thus more valuable in downstream operations, such as adjusting the syngas ratio of an H₂/CO stream for possible use in Fischer-Tropsch or methanol processes.

The present catalysts are preferably provided in the form of foam, monolith, gauze, noodles, balls, pills, granules, spheres, beads, pellets, trilobes or the like, for operation at the desired high gas velocities with minimal back pressure. While the catalysts can be self-supporting, they are preferably provided as a surface layer on a support.

Examples of suitable refractory supports include cordierite, cordierite-alpha alumina, silicon nitride, zircon mullite, spodumene, alumina-silica magnesia, zircon silicate, sillimanite, magnesium silicates, zircin, petalite, alpha and gamma aluminas and aluminosilicates which may be amorphous or crystalline (i.e. alumina-zirconia, alumina-chromia, alumina-ceria, etc.), zirconia, magnesium stabilized zirconia, zirconia stabilized alumina, yttrium stabilized zirconia, calcium stabilized zirconia, titania, silica, magnesia, niobia and vanadia, carbon black, CaCO₃, BaSO₄, silica-alumina, and alumina.

In some embodiments, ODH is carried out using the hydrocarbon feed mixed with an appropriate oxidant and possibly steam. Appropriate oxidants may include, but are not limited to I₂, O₂, N₂O, CO₂ and SO₂. Use of the oxidant shifts the equilibrium of the dehydrogenation reaction toward complete conversion through formation of compounds containing the abstracted hydrogen (e.g. H₂O, HI, H₂S). Steam, on the other hand, may be used to activate the catalyst, remove coke from the catalyst, or serve as a diluent for temperature control.

Process Conditions

Any suitable reaction regime is applied in order to contact the reactants with the catalyst. One suitable regime is a fixed bed reaction regime, in which the catalyst is retained within a reaction zone in a fixed arrangement. Catalysts may be employed in the fixed bed regime, retained using fixed bed reaction techniques well known in the art. Preferably a millisecond contact time reactor is employed. A general description of major considerations involved in operating a reactor using millisecond contact times is given in U.S. Pat. No. 6,072,097, which is incorporated herein by reference.

Accordingly, a feed stream comprising a hydrocarbon feedstock and an oxygen-containing gas is contacted with one of the above-described non-precious metal oxide catalysts in a reaction zone maintained at conversion-promoting conditions effective to produce an effluent stream comprising alkenes. The hydrocarbon feedstock may be any gaseous hydrocarbon having a low boiling point, such as ethane, natural gas, associated gas, or other sources of light hydrocarbons having from 1 to 10 carbon atoms. In addition, hydrocarbon feeds including naphtha and similar feeds may be employed. The hydrocarbon feedstock may be a gas arising from naturally occurring reserves of ethane, which contain carbon dioxide. Preferably, the feed comprises at least 50% by volume alkanes (<C₁₀).

The hydrocarbon feedstock is contacted with the catalyst as a gaseous phase mixture with an oxygen-containing gas, preferably pure oxygen. The oxygen-containing gas may also comprise steam and/or CO₂ in addition to oxygen. Alternatively, the hydrocarbon feedstock is contacted with the catalyst as a mixture with a gas comprising steam and/or CO₂.

The process is operated at atmospheric or superatmospheric pressures, the latter being preferred. The pressures may be from about 100 kPa to about 32,500 kPa, preferably from about 130 kPa to about 12,500 kPa. While the preheat in prior art occurs in the range of 0° C. to 500° C. and typically 25° C. to 400° C., the preheat temperature of the present invention occurs at temperatures of from about 300° C. to about 800° C., preferably from about 350° C. to about 700° C. The hydrocarbon feedstock and the oxygen-containing gas are preferably pre-heated before contact with the catalyst. The hydrocarbon feedstock and the oxygen-containing gas are passed over the catalyst at any of a variety of space velocities.

Gas hourly space velocities (GHSV) for the present process, stated as normal liters of gas per kilogram of catalyst per hour, are from about 20,000 to at least about 100,000,000 NL/kg/h, preferably from about 50,000 to about 50,000,000 NL/kg/h. Preferably the catalyst is employed in a millisecond contact time reactor. The process preferably includes maintaining a catalyst residence time of no more than 200 milliseconds for the reactant gas mixture. Residence time is inversely proportional to space velocity, and high space velocity indicates low residence time on the catalyst. An effluent stream of product gases, including alkenes, alkynes, CO, CO₂, H₂, H₂O, and unconverted alkanes emerges from the reactor.

In some embodiments, unconverted alkanes may be separated from the effluent stream of product gases and recycled back into the feed. Product H₂ and CO may be recovered and used in other processes such as Fischer-Tropsch synthesis and methanol production.

In some embodiments the use of steam may be employed. As mentioned above, steam may be used to activate the catalyst, remove coke from the catalyst, or serve as a diluent for temperature control.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. For example, the present invention may be incorporated into a gas to liquids plant (GTL) or methanol plant or may stand alone. Accordingly, the scope of protection is not limited by the description set out above, but is only limited by the claims which follow, that scope including all equivalents of the subject matter of the claims. The disclosures of all patents and publications cited herein are incorporated by reference in their entireties.

Catalysts

In the following examples, the refractory supports were purchased from Porvair Advanced Materials. The base metal, metal oxide, and base metal-metal oxide coatings were added by an incipient wetness technique, wherein incipient wetness of the supports was achieved using aqueous solutions of a soluble metal salts such as nitrate, acetate, chlorides, acetylacetonate or the like. All results are at C₂H₆:O₂ of 1.8. For prior art catalysts, comparative results of 2 wt % Pt/Al₂O₃ taken from U.S. Pat. No. 6,072,097 with C₂H₆:O₂ of 1.9. The final catalysts test in the form of foam monoliths.

Test Procedure and Results

Once the catalysts were produced, they were tested in an atmospheric millisecond contact time reactor at 900,000 NL/kg/h using an ethane feed with a 10% nitrogen dilution and a molar fuel to oxygen ratio of 1.8. This affords a reactant gas to catalyst contact time of 10 to 30 milliseconds. The results can be seen in Table 1 below.

TABLE 1
Test Results

| | Preheat | | Ethylene | CO | H₂ | | | |
|---|---|---|---|---|---|---|---|---|
| Catalyst | Temp | Ethane | Selec- | Selec- | Selec- | Ethylene | CO | H₂ |
| Description | (° C.) | Conv. | tivity | tivity | tivity | Yield | Yield | Yield |
| 2 wt % Pt/Al₂O₃* | NA | 70 | 65 | 27 | NM | 45 | 21 | 20 |
| 1.4 wt % Pt | 350 | 90 | 61 | 25 | 24 | 55 | 23 | 22 |
| 0.3 wt % Au/PSZ* | | | | | | | | |
| 0.7 wt % Sn/PSZ | 600 | 86 | 65 | 20 | 25 | 56 | 17 | 22 |
| 2.4 wt % Sn/PSZ | 665 | 95 | 55 | 21 | 27 | 52 | 20 | 26 |
| 1.5 wt % Fe/Al₂O₃ | 525 | 80 | 68 | 18 | 20 | 54 | 16 | 16 |
| 1.7 wt % Fe/PSZ | 600 | 83 | 62 | 21 | 29 | 52 | 17 | 24 |
| 1.7 wt % Cr/Al₂O₃ | 525 | 82 | 67 | 19 | 23 | 55 | 16 | 20 |
| 2.7 wt % Cr/Al₂O₃ | 525 | 85 | 70 | 13 | 25 | 60 | 11 | 21 |
| 1.4 wt % Cu/PSZ | 525 | 92 | 61 | 19 | 25 | 56 | 18 | 23 |
| 1.9 wt % Mn/Al₂O₃ | 600 | 87 | 64 | 20 | 25 | 56 | 17 | 22 |
| 2.1 wt % Au/PSZ | 600 | 95 | 55 | 17 | 21 | 53 | 17 | 20 |
| 3.0 wt % Ni/Al₂O₃ | 650 | 82 | 58 | 34 | 43 | 47 | 28 | 35 |
| 5.4 wt % Sm/Al₂O₃ | 525 | 95 | 47 | 27 | 26 | 45 | 26 | 25 |
| 1.5 wt % Co/Al₂O₃ | 600 | 77 | 54 | 35 | 47 | 41 | 27 | 36 |

*Comparative results of 2 wt % Pt/Al₂O₃ taken from U.S. Pat. No. 6,072,097 with C₂H₆:O₂ of 1.9.
NA = Not Applicable.
NM = Not Measured.

From the results, it can be seen that non-precious base metals, metal oxides, or combinations thereof placed on refractory supports can be made to produce ethylene yields comparable to previously known precious metal-containing supports.

What is claimed is:

1. A catalyst for use in oxidative dehydrogenation processes comprising:
a refractory support, and
a base metal selected from the group consisting of Group IB-VIIB metals, Group IIIA-VA metals, Lanthanide metals, iron, cobalt, and nickel, or a metal oxide selected from the group consisting of alumina, stabilized aluminas, zirconia, stabilized zirconias, titania, ytteria, silica, niobia, and vanadia, or a combination of a base metal and a metal oxide;
wherein the base metal, metal oxide, or combination thereof is coated on the refractory support.

2. The catalyst of claim 1 wherein the metal oxide consists essentially of stabilized zirconia.

3. The catalyst of claim 1 wherein the catalyst is calcined at 300-1200° C.

4. The catalyst of claim 1 wherein the catalyst is calcined for 1-12 hours.

5. The catalyst of claim 1 wherein ethylene yield is at least 25%.

6. The catalyst of claim 1 wherein ethylene yield is at least 40%.

7. A method for the production of olefins comprising:
heating a feed stream comprising an alkane and an oxidant stream to a temperature of approximately 300-700° C.;
contacting said alkane and oxidant stream with a catalyst comprising a refractory support and a base metal, metal oxide, or a combination thereof;
maintaining a contact time of said alkane with said catalyst for less than 200 milliseconds; and
maintaining oxidative dehydrogenation favorable conditions.

8. The method of claim 7 wherein the oxidant consists essentially of pure oxygen.

9. The method of claim 7 wherein the base metal is selected from the group consisting of Group IB-VIIB metals, Group IIIA-VA metals, Lanthanide metals, iron, cobalt and nickel.

10. The method of claim 7 wherein the metal oxide is selected from the group consisting of alumina, stabilized alumina, zirconia, stabilized zirconias, titania, ytteria, silica, niobia, and vanadia.

11. The method of claim 10 wherein the metal oxide consists essentially of stabilized zirconia.

12. The method of claim 7 wherein said feed stream is heated to a t least about 500° C.

13. The method of claim 7 wherein ethylene yield is at least 25%.

14. The method of claim 7 wherein ethylene yield is at least 40%.

15. A method for converting alkanes to olefins comprising:
heating a feed stream comprising an alkane and an oxidant to a temperature of approximately 300-700° C.;
contacting said feed stream with a catalyst comprising a base metal, metal oxide, or a combination thereof and a refractory support;
maintaining a contact time of said alkane with said catalyst for less than 200 milliseconds; and
maintaining oxidative dehydrogenation favorable conditions.

16. The method of claim 15 wherein the oxidant consists essentially of pure oxygen.

17. The method of claim 15 wherein the base metal is selected from the group consisting of Group IB-VIIB metals, Group IIIA-VA metals, Lanthanide metals, iron, cobalt and nickel.

18. The method of claim 15 wherein the metal oxide is selected from the group consisting of alumina, stabilized aluminas, zirconia, stabilized zirconias, titania, ytteria, silica, niobia, and vanadia.

19. The method of claim 18 wherein the metal oxide consists essentially of stabilized zirconia.

20. The method of claim 15 wherein said feed stream is heated to at least about 500° C.

21. The method of claim 15 wherein ethylene yield is at least 25%.

22. The method of claim 15 wherein ethylene yield is at least 40%.

23. An oxidative dehydrogenation catalyst comprising:
a base metal selected from the group consisting of Group IB-VIIB metals, Group IIIA-VA metals, Lanthanide metals, iron, cobalt, and nickel, or a metal oxide selected from the group consisting of alumina, zirconia, stabilized zirconias, titania, and ytteria, or a combination of a base metal and a metal oxide; and
a refractory support,
wherein the base metal, metal oxide, or combination thereof is coated on the refractory support.

24. The catalyst of claim 23 wherein the metal oxide consists essentially of stabilized zirconia.
